(19) United States
(12) Patent Application Publication
(10) Pub. No.: US 2019/0065489 A1
(43) Pub. Date: Feb. 28, 2019

(54) METHOD AND SYSTEM FOR ASSIGNING, ROUTING, AND UNASSIGNING DATA FLOWS OF ULTRASOUND PATCH PROBES

(71) Applicant: General Electric Company, Schenectady, NY (US)

(72) Inventor: Menachem Halmann, Wauwatosa, WI (US)

(21) Appl. No.: 15/685,761

(22) Filed: Aug. 24, 2017

(51) Int. Cl. G06F 17/30; A61B 8/08; G06F 19/28

(52) U.S. Cl. CPC G06F 17/303; G06F 17/3028; G06F 19/28; A61B 8/52

(57) ABSTRACT

An ultrasound patch probe having ultrasound image acquisition functionality is selectively placed on a patient in communication range of an ultrasound imaging system. A signal processor of the ultrasound imaging system detects a presence of the ultrasound patch probe to establish a connection between the ultrasound patch probe and the ultrasound imaging system. The signal processor assigns the detected ultrasound patch probe to one or both of a patient and an anatomy of the patient. The signal processor maps the ultrasound patch probe to ultrasound image routing parameters based on the assignment. The signal processor routes ultrasound image data acquired by the ultrasound patch probe to one or both of a display area of a display system for presentation of the acquired ultrasound image data and a storage location of a data storage medium for storing the acquired ultrasound image data based on the ultrasound image routing parameters.

### CROSS-REFERENCE TO RELATED APPLICATIONS/INCORPORATION BY REFERENCE

[Not Applicable]

### FIELD

Certain embodiments of the disclosure relate to ultrasound imaging. More specifically, certain embodiments of the disclosure relate to a method and system for assigning, routing, and unassigning data flows of ultrasound patch probes.

### BACKGROUND

Ultrasound imaging is a medical imaging technique for imaging organs and soft tissues in a human body. Ultrasound imaging uses real time, non-invasive high frequency sound waves to produce a two-dimensional (2D) image and/or a three-dimensional (3D) image. Ultrasound examinations are typically performed by an ultrasound operator placing an ultrasound transducer on a body surface and manipulating the ultrasound transducer about the body surface to manually control the acquisition of ultrasound image data. The manual manipulation of the ultrasound transducer is not ideal for prolonged ultrasound image data acquisition. Instead, an ultrasound patch probe may be secured in a fixed position on the body surface of a patient for ultrasound image data acquisition over an extended period of time. In certain scenarios, ultrasound systems may be capable of simultaneously acquiring ultrasound image data from multiple ultrasound probes, such as patch probes, that may be connected to the ultrasound system. The ultrasound patch probes connected to an ultrasound system may be secured to different anatomy of a patient and/or to different patients. In some cases, it may be difficult distinguishing between the data flows of the different ultrasound patch probes connected to an ultrasound system.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method for assigning, routing, and unassigning data flows of ultrasound patch probes is provided, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system having one or more ultrasound patch probes, in accordance with various embodiments.

FIG. 2 is a flow chart illustrating exemplary steps that may be utilized for assigning, routing, and unassigning data flows of ultrasound patch probes, in accordance with various embodiments.

### DETAILED DESCRIPTION

Certain embodiments may be found in a method and system for assigning, routing, and unassigning data flows of ultrasound patch probes. For example, aspects of the present disclosure have the technical effect of assigning detected ultrasound patch probes to one or both of a patient and an anatomy of a patient. Moreover, aspects of the present disclosure have the technical effect of mapping ultrasound patch probes to ultrasound scanning parameters and/or ultrasound image data routing parameters based on an assignment of detected patch probes to a patient and/or patient anatomy. Additionally, aspects of the present disclosure have the technical effect of continuously acquiring and routing ultrasound image data from ultrasound patch probes based on a mapping of the ultrasound patch probes to ultrasound scanning parameters and/or ultrasound image data routing parameters. Furthermore, aspects of the present disclosure have the technical effect of detecting conditions for unassigning an ultrasound patch probe such that non-relevant ultrasound data is not routed to a display, report, archive, and/or the like.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments of the present disclosure. The following detailed description is, therefore, not to be taken in a limiting sense, and the scope of the present disclosure is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode, CF-mode and/or sub-modes of CF such as TVI, Angio, B-flow, BMI, BMI_Angio, and in some cases also MM, CM, PW, TVD, CW where the “image” and/or “plane” includes a single beam or multiple beams.

Furthermore, the term processor or processing unit, as used herein, refers to any type of processing unit that can carry out the required calculations needed for the disclosure, such as single or multi-core: CPU, Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

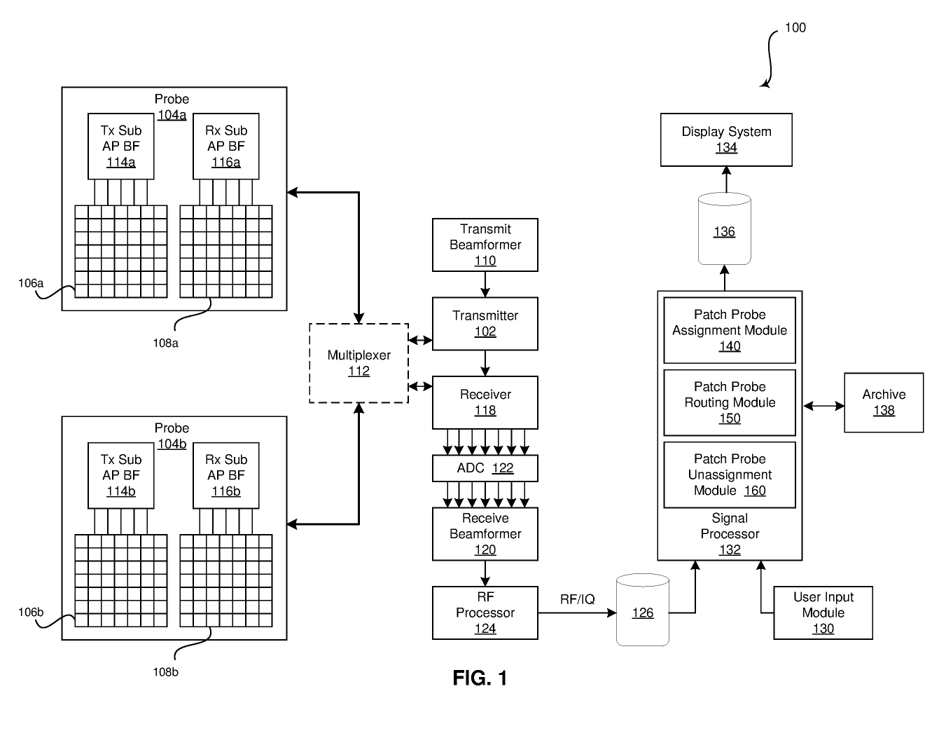

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 having one or more ultrasound patch probes 104a, 104b, in accordance with various embodiments. Referring to FIG. 1, there is shown an ultrasound system 100 comprising a transmitter 102, ultrasound probes 104a, 104b, a transmit beamformer 110, a multiplexer 112, a receiver 118, a receive beamformer 120, a RF processor 124, a RF/IQ buffer 126, a user input module 130, a signal processor 132, an image buffer 136, a display system 134, and an archive 138.

The transmitter 102 may comprise suitable logic, circuitry, interfaces and/or code that may be operable to drive one or more ultrasound probes 104a, 104b. The ultrasound probe(s) 104a, 104b may comprise a one dimensional (1D, 1.25D, 1.5D or 1.75D) array or two dimensional (2D) array of piezoelectric elements. The ultrasound probe(s) 104a, 104b may each comprise a group of transmit transducer elements 106a, 106b and a group of receive transducer elements 108a, 108b, that normally constitute the same elements. The ultrasound probe(s) 104a, 104b may include one or more ultrasound patch probes and/or one or more manually manipulated ultrasound probes. In various embodiments, an ultrasound operator may attach ultrasound patch probes 104a, 104b to different patients and/or different patient anatomy. Although certain embodiments may illustrate and/or describe two ultrasound probes 104a, 104b, for example, unless so claimed, the scope of various aspects of the present disclosure should not be limited to using two ultrasound probes 104a, 104b and may additionally and/or alternatively be applicable to any suitable number of ultrasound probes 104a, 104b connected to the ultrasound system 100. For example, certain embodiments provide more or less than the two ultrasound probes 104a, 104b illustrated in FIG. 1. In various embodiments, with respect to the array(s) of transducer elements 106a, 106b, 108a, 108b in probes 104a, 104b, a variety of different geometries and configuration may be used and the transducer elements 106a, 106b, 108a, 108b may be provided as part of, for example, different types of ultrasound probes. In certain embodiments, one or more of the transducer elements 106a, 106b, 108a, 108b may be configured having the same geometry, for example, the same size or configuration and may be part of the same type of ultrasound probe. In an exemplary embodiment, the ultrasound probe(s) 104a, 104b may be configured to communicate with transmitter 102 and receiver 118 via wired and/or wireless communication. For example, the ultrasound probe(s) 104a, 104b may be configured to transmit and receive signals using near field communication (NFC), Bluetooth, Wi-Fi, or any suitable wireless technology.

The transmit beamformer 110 may comprise suitable logic, circuitry, interfaces and/or code that may be operable to control the transmitter 102 which, through a transmit sub-aperture beamformer 114a, 114b, drives the group of transmit transducer elements 106a, 106b to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108a, 108b. The group of receive transducer elements 108a, 108b in each of the ultrasound probes 104a, 104b may be operable to convert the received echoes into analog signals, undergo sub-aperture beamforming by a receive sub-aperture beamformer 116a, 116b and are then communicated to a receiver 118.

The multiplexer 112 may comprise suitable logic, circuitry, interfaces and/or code that may be operable to control which of the one or more transmit transducer elements 106a, 106b and/or ultrasound probes 104a, 104b is driven, including the specific elements within a particular array of transmit transducer elements 106a, 106b. The multiplexer 112 may comprise suitable logic, circuitry, interfaces and/or code that may be operable to multiplex the echoes received from the receive transducer elements 108a, 108b via the receive sub-aperture beamformers 116a, 116b when using more than one ultrasound probe 104a, 104b and corresponding array of receive transducer elements 108a, 108b. In various embodiments, the multiplexer 112 and other switching circuitry may be provided in the ultrasound probe housing 104a, 104b and/or the housing of the ultrasound system 100. Additionally and/or alternatively, certain embodiments provide that multiple ultrasound patch probes 104a, 104b may be driven in parallel and the data flows from the multiple ultrasound patch probes 104a, 104b may be processed in parallel without multiplexing.

The receiver 118 may comprise suitable logic, circuitry, interfaces and/or code that may be operable to receive and demodulate the signals from the receive sub-aperture beamformer(s) 116a, 116b. The demodulated analog signals may be communicated to one or more of the plurality of A/D converters 122. The plurality of A/D converters 122 may comprise suitable logic, circuitry, interfaces and/or code that may be operable to convert the demodulated analog signals from the receiver 118 to corresponding digital signals. The plurality of A/D converters 122 are disposed between the receiver 118 and the receive beamformer 120. Notwithstanding, the disclosure is not limited in this regard. Accordingly, in some embodiments, the plurality of A/D converters 122 may be integrated within the receiver 118.

The receive beamformer 120 may comprise suitable logic, circuitry, interfaces and/or code that may be operable to perform digital beamforming processing on the signals received from the plurality of A/D converters 122. The resulting processed information may be converted back to corresponding RF signals. The corresponding output RF signals that are output from the receive beamformer 120 may be communicated to the RF processor 124. In accordance with some embodiments, the receiver 118, the plurality of A/D converters 122, and the beamformer 120 may be integrated into a single beamformer, which may be digital.

The RF processor 124 may comprise suitable logic, circuitry, interfaces and/or code that may be operable to demodulate the RF signals. In accordance with an embodiment, the RF processor 124 may comprise a complex demodulator (not shown) that is operable to demodulate the RF signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The user input module may be utilized to input patient data, image acquisition and scan parameters, image viewing parameters, settings, configuration parameters, change scan mode, start and stop scanning, assign detected ultrasound probe(s) 104a, 104b data flows to a patient and/or a patient anatomy, unassign ultrasound probe(s) 104a, 104b data flows, and the like. In an exemplary embodiment, the user input module 130 may be operable to configure, manage and/or control operation of one or more components and/or modules in the ultrasound system 100. In this regard, the user input module 130 may be operable to configure, manage and/or control operation of transmitter 102, the ultrasound probe(s) 104a, 104b, the transmit beamformer 110, the receiver 118, the receive beamformer 120, the A/D converters 122, the RF processor 124, the RF/IQ buffer 126, the user input module 130, the signal processor 132, the image buffer 136, and/or the display system 134.

The user input module 130 may include physical control devices provided and/or integrated at the ultrasound system 100. For example, the user input module 130 can include a trackball, mousing device, keyboard, touch screen display, remote control, button, switch, rotary encoder, sliding bar, and/or voice activated input, among other things. The user input module 130 may be integrated with other components, such as the ultrasound probe(s) 104a, 104b, display system 134 or control panel, or can be a separate component.

The signal processor 132 may comprise suitable logic, circuitry, interfaces and/or code that may be operable to control operation of the ultrasound system 100 and process the ultrasound scan data (i.e., RF signal data or IQ data pairs) for generating an ultrasound image for presentation on a display system 134. The signal processor 132 is operable to perform one or more processing operations according to a plurality of selectable ultrasound modalities on the acquired ultrasound scan data. In an exemplary embodiment of the disclosure, the signal processor 132 may be operable to perform volume rendering, compounding, motion tracking, and/or speckle tracking. Acquired ultrasound scan data may be processed in real-time during a scanning session as the echo signals are received. Additionally or alternatively, the ultrasound scan data may be stored temporarily in the RF/IQ buffer 126 and/or the archive 138 during a scanning session and processed in less than real-time in a live or off-line operation. The processed image data can be presented at the display system 134, inserted into a report, and/or stored at the archive 138. The archive 138 may be a local archive, a Picture Archiving and Communication System (PACS), or any suitable device for storing images and related information. In an exemplary embodiment, the signal processor 132 may comprise a patch probe assignment module 140, a patch probe routing module 150, and a patch probe unassignment module 160.

The ultrasound system 100 may be operable to continuously acquire ultrasound information at a frame rate that is suitable for the imaging situation in question. Typical frame rates range from 20-70 but may be lower or higher. The acquired ultrasound information may be displayed on the display system 134 at a display-rate that can be the same as the frame rate, or slower or faster. An image buffer 136 is included for storing processed frames of acquired ultrasound information that are not scheduled to be displayed immediately. Preferably, the image buffer 136 is of sufficient capacity to store at least several seconds worth of frames of ultrasound information. The frames of ultrasound information are stored in a manner to facilitate retrieval thereof according to its order or time of acquisition. The image buffer 136 may be embodied as any known data storage medium.

The patch probe assignment module 140 may comprise suitable logic, circuitry, interfaces and/or code that may be operable to detect ultrasound probe(s) 104a, 104b attempting to connect with the ultrasound system 100. For example, an ultrasound probe 104a, 104b may transmit a signal via wired or wireless communication to attempt to connect with an ultrasound system 100. The ultrasound system 100 may receive the transmitted signal and provide it to the patch probe assignment module 140 of the signal processor 132. The patch probe assignment module 140 may decode the received signal and analyze the received information provided in the signal to determine whether an authorized ultrasound probe 104a, 104b configured to connect to the ultrasound system 100 has been detected. The information provided in the signal may include identification information and the type of ultrasound probe 104a, 104b, among other things. If the patch probe assignment module 140 determines that the received signal corresponds to an ultrasound probe 104a, 104b capable of and/or authorized to connect to the ultrasound system 100, the patch probe assignment module 140 may be configured to assign the detected ultrasound probe 104a, 104b to a patient and/or an anatomy of the patient. The patch probe assignment module 140 may also map the assigned ultrasound probe 104a, 104b to ultrasound scanning parameters and/or ultrasound image data routing parameters based on the assignment.

In various embodiments, the patch probe assignment module 140 may assign a detected ultrasound probe 104a, 104b to a patient and/or an anatomy of the patient based on stored settings associated with the particular ultrasound probe 104a, 104b, user instructions, or the like. For example, the patch probe assignment module 140 may provide an assignment dialog user interface to allow the ultrasound operator to configure the detected ultrasound probe 104a, 104b. The ultrasound operator may provide text, button selections, drop-down menu selections, and/or the like via the assignment dialog user interface to instruct the patch probe assignment module 140 with respect to an identification of an associated patient and/or an anatomy of the patient to which the detected ultrasound probe 104a, 104b is attached.

The patch probe assignment module 140 may map the assigned ultrasound probe 104a, 104b to ultrasound scanning parameters based on the assignment. For example, different applications of an ultrasound patch probe 104a, 104b may involve different scanning parameters. The scanning parameters may define an acquisition mode, pulse repetition frequency (PRF), depth, gain, ultrasound frequency, focal zone, and the like. As an example, an ultrasound patch probe 104a, 104b monitoring a flow in the carotid artery may include Doppler and/or Color Flow modes and a pulse repetition frequency (PRF) parameter set to a relative high velocity of carotid flow. As another example, a B mode optimized to an area below a pleura level with a frequency optimized based on depth may be selected for an ultrasound patch probe 104a, 104b scanning the lungs for signs of Pneumothorax, Pulmonary Edema, or Atelectasis. In various embodiments, the patch probe assignment module 140 may map an ultrasound patch probe 104a, 104b to a predefined set of ultrasound scanning parameters based on the assignment of the probe 104a, 104b to a particular patient anatomy, such as the carotid artery, lungs, or any suitable patient anatomy. The predefined sets of ultrasound scanning parameters may be stored at archive 138 or any suitable data storage medium. The ultrasound scanning parameters are executed by the ultrasound patch probe 104a, 104b during operation of the probe 104a, 104b to acquire ultrasound image data.

The patch probe assignment module 140 may map the assigned ultrasound probe 104a, 104b to ultrasound image data routing parameters based on the assignment. For example, routing parameters may include an archive storage destination, a display system presentation destination, and/or a report placement destination, among other things. Specifically, the routing parameters may define where in an archive 138 ultrasound image data corresponding with a particular patient and/or a specific anatomy of a particular patient should be stored. The routing parameters may define where in a display area of a display system 134 ultrasound image data corresponding with a particular patient and/or a specific anatomy of a particular patient should be presented. The routing parameters may define a particular report and/or a specific placement within a particular report that ultrasound image data corresponding with a particular patient and/or a specific anatomy of a particular patient should be inserted. The routing parameters assist an operator with identifying the ultrasound image data acquired via each ultrasound probe 104a, 104b, particularly in situations where multiple probes 104a, 104b are operating to simultaneously acquire ultrasound image data. The routing parameters may be stored at archive 138 or any suitable data storage medium. The routing parameters are executed by the patch probe routing module 150 of the signal processor 132 as ultrasound image data is received at the signal processor 132 from one or more ultrasound probes 104a, 104b.

The patch probe routing module 150 may comprise suitable logic, circuitry, interfaces and/or code that may be operable to route received ultrasound image data from one or more ultrasound probes 104a, 104b based on the mapping. For example, routing parameters stored in connection with the mapping of an ultrasound probe 104a, 104b may be retrieved and executed to determine where the received ultrasound image data associated with a particular probe 104a, 104b is to be stored, displayed, and/or inserted into a report, among other things. The patch probe routing module 150 may analyze the received ultrasound image data to determine the associated ultrasound probe 104a, 104b that corresponds to the particular data flow. The patch probe routing module 150 may retrieve the appropriate routing parameters from the archive 138 or any suitable data storage medium based on the identification of the data flow. The patch probe routing module 150 executes the retrieved routing parameters to route the data flow to the appropriate location(s). In various embodiments, the patch probe routing module 150 may route data flows acquired substantially simultaneously from multiple patch probes and/or from patch probe(s) and manual scanning probe(s).

The patch probe unassignment module 160 may comprise suitable logic, circuitry, interfaces and/or code that may be operable to stop the acquisition and/or routing of an ultrasound image data flow in response to a received indication for unassignment of an ultrasound probe 104a, 104b. The indication for unassignment may include, for example, a received operator instruction to unassign a probe 104a, 104b, a disconnection of the ultrasound probe 104a, 104b from the ultrasound system 100, a detected air-scanning condition of a patch probe 104a, 104b, and/or a detected patch probe movement condition, among other things.

For example, the patch probe unassignment module 160 may provide an assignment dialog user interface to allow the ultrasound operator to deactivate an assigned ultrasound probe 104a, 104b. As another example, the patch probe unassignment module 160 may detect if a wired ultrasound probe 104a, 104b is unplugged from the ultrasound system 100 or if a wireless ultrasound probe 104a, 104b is moved out of range of the ultrasound system 100. In an exemplary embodiment, the patch probe unassignment module 160 may detect an air-scanning condition of a patch probe 104a, 104b if, for example, the patch probe unassignment module 160 applies image analysis algorithms that determine the acquired ultrasound image data is static over a predetermined period of time (i.e., no changes from frame to frame) and/or if the patch probe unassignment module 160 applies image analysis algorithms that determine the only received reflections are from superficial gel. The air-scanning condition identifies that an ultrasound patch probe has become unsecured from the patient and is not acquiring relevant ultrasound image data. As a further example, the patch probe unassignment module 160 may detect that a patch probe 104a, 104b has moved relative to the surface of the skin of the patient and is no longer acquiring ultrasound image data of the appropriate patient anatomy by analyzing the superficial area of the acquired ultrasound image data and comparing sequential frames using cross-correlation algorithms to detect such motion. Moreover, the patch probe unassignment module 160 may detect that a patch probe 104a, 104b has moved relative to the surface of the skin of the patient by analyzing motion sensor data received from motion sensors attached to the ultrasound patch probe 104a, 104b and the patient. For example, the detection of significant motion at the ultrasound patch probe 104a, 104b that is not detected at a motion sensor attached to the patient may indicate that the patch probe 104a, 104b has moved.

The patch probe unassignment module 160 may stop acquiring and/or stop routing ultrasound image data from the unassigned ultrasound patch probe in response to the received indication for unassignment of the ultrasound probe 104a, 104b. For example, the patch probe unassignment module 160 may continue acquiring but may stop routing ultrasound image data flows from ultrasound patch probes 104a, 104b where an air scanning or probe movement condition has been identified. In various embodiments, the patch probe unassignment module 160 may re-assign the patch probe 104a, 104b such that the patch probe routing module 150 may resume routing the data flow if the patch probe 104a, 104b is reattached and/or repositioned within a predetermined period of time. In certain embodiments, the patch probe unassignment module 160 may otherwise turn off and/or disconnect the patch probe 104a, 104b after a predetermined period of time of continuous air scanning or non-resolved probe movement. As another example, the patch probe unassignment module 160 may cause the unassigned ultrasound probe 104a, 104b to stop scanning and simultaneously cause the patch probe routing module 150 to stop routing data flows associated with the unassigned ultrasound probe 104a, 104b. Additionally and/or alternatively, the patch probe unassignment module 160 may cause the patch probe routing module 150 to stop routing data flows associated with the unassigned ultrasound probe 104a, 104b without turning off or otherwise preventing the unassigned ultrasound probe 104a, 104b from acquiring ultrasound data. In various embodiments, an unassigned ultrasound probe 104a, 104b may be reconnected and/or reassigned by the patch probe assignment module 140 after the probe 104a, 104b is unassigned by the patch probe unassignment module 160 or after the probe 104a, 104b is unassigned by the patch probe unassignment module 160 and a predetermined period of time has elapsed.

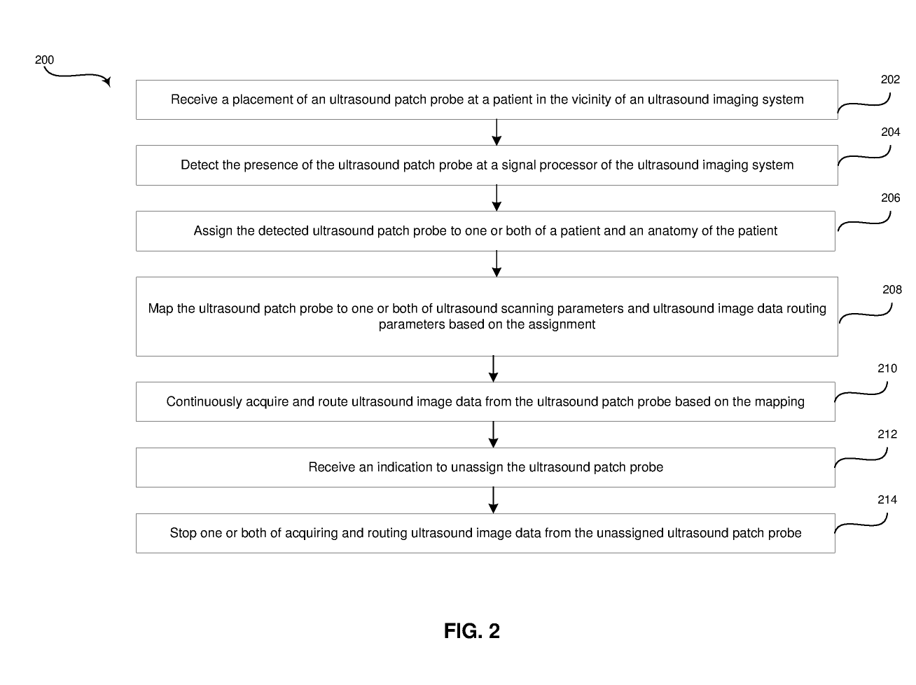

FIG. 2 is a flow chart illustrating exemplary steps 202-214 that may be utilized for assigning, routing, and unassigning data flows of ultrasound patch probes 104a, 104b, in accordance with various embodiments. Referring to FIG. 2, there is shown a flow chart 200 comprising exemplary steps 202 through 214. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 202, an ultrasound patch probe 104a, 104b may be positioned on a patient anatomy in the vicinity of an ultrasound imaging system 100. For example, an ultrasound imaging system operator may position and detachably couple an ultrasound patch probe 104a, 104b at a location on the skin of a patient for prolonged monitoring of a particular patient anatomy, such as the left common carotid artery, the posterior side of the left lung, or any suitable patient anatomy. The ultrasound patch probe 104a, 104b may be secured to the skin of a patient at a desired location by an adhesive or any suitable positioning mechanism. The ultrasound patch probe 104a, 104b may be wired or wireless to provide acquired ultrasound image data to the nearby ultrasound imaging system 100.

At step 204, a signal processor 132 of an ultrasound imaging system 100 may detect the presence of the ultrasound patch probe 104a, 104b positioned on the patient anatomy at step 202. For example, the ultrasound patch probe 104a, 104b may transmit a signal to attempt to connect with the ultrasound imaging system 100. The ultrasound imaging system 100 may receive the transmitted signal via wired or wireless communications. For example, an ultrasound imaging system operator may plug an ultrasound patch probe 104a, 104b into the ultrasound imaging system 100 such that the ultrasound patch probe 104a, 104b may transmit the signal via a wire, cable, or the like to the ultrasound imaging system 100. As another example, the ultrasound patch probe 104a, 104b may include a transceiver operable to transmit signals to and receive signals from a transceiver of the ultrasound imaging system 100. The signals received at the transceiver of the ultrasound imaging system 100 from the ultrasound probe 104a, 104b may be provided to the signal processor 132 of the ultrasound imaging system 100. A patch probe assignment module 140 of the signal processor 132 may analyze information provided in the received signal, such as identification information and the type of ultrasound probe, to determine whether an authorized ultrasound probe 104a, 104b has been detected.

At step 206, the patch probe assignment module 140 of the signal processor 132 may assign the ultrasound probe 104a, 104b detected at step 204 to one or both of a patient and an anatomy of the patient. For example, the patch probe assignment module 140 of the signal processor 132 may assign the detected ultrasound probe 104a, 104b based on stored settings associated with the particular ultrasound probe 104a, 104b or instructions provided by an operator of the ultrasound imaging system 100, among other things. In various embodiments, the patch probe assignment module 140 may provide an assignment dialog user interface to allow the ultrasound operator to provide text, button selections, drop-down menu selections, and/or the like to configure the detected ultrasound probe 104a, 104b. The ultrasound operator may configure the ultrasound probe 104a, 104b by providing instructions to the patch probe assignment module 140 associating the detected ultrasound probe 104a, 104b to a particular patient and/or a specifically identified anatomy of the patient to which the detected ultrasound probe 104a, 104b is attached. In various embodiments, names, numbers, codes, and/or the like may be used to identify each particular patient and each anatomical option. For example, an ultrasound patch probe 104a, 104b assigned to patient number 1234 placed on the left common carotid artery may be assigned as PID1234, LeftCCA.

At step 208, the patch probe assignment module 140 of the signal processor 132 may map the ultrasound probe 104a, 104b to ultrasound scanning parameters and/or ultrasound data routing parameters based on the assignment at step 206. For example, sets of scanning parameters each having a predefined relationship with a patient anatomy may be stored at archive 138 or any suitable data storage medium. The patch probe assignment module 140 may map the appropriate set of scanning parameters to an assigned ultrasound probe 104a, 104b based at least in part on the assigned anatomy of the patient. The assigned ultrasound probe 104a, 104b may execute the mapped set of scanning parameters during operation of the assigned ultrasound probe 104a, 104b to acquire ultrasound image data. As another example, the patch probe assignment module 140 may map routing parameters that define a location in an archive 138 for storing ultrasound image data corresponding with the assigned ultrasound probe 104a, 104b. The patch probe assignment module 140 may map routing parameters that define a display area of a display system 134 for presenting ultrasound image data corresponding with the assigned ultrasound probe 104a, 104b. The patch probe assignment module 140 may map routing parameters that define a particular report and/or a specific placement within a particular report for inserting ultrasound image data corresponding with the assigned ultrasound probe 104a, 104b. The routing parameters may be stored at archive 138 or any suitable data storage medium for retrieval and execution by the signal processor 132 to route the data flow received from the mapped ultrasound probe 104a, 104b.

At step 210, the ultrasound imaging system 100 may continuously acquire and route ultrasound image data from the ultrasound probe 104a, 104b based on the mapping at step 208. For example, the ultrasound probe 104a, 104b, under the control of the ultrasound imaging system 100, may continuously acquire ultrasound image data. The data flow from the ultrasound probe 104a, 104b may be received at the signal processor 132 and evaluated by a patch probe routing module 150 of the signal processor 132 to determine the associated ultrasound probe 104a, 104b corresponding with the particular data flow. The patch probe routing module 150 retrieves the routing parameters corresponding with the identified data flow from the archive 138 or any suitable data storage medium. The retrieved routing parameters are executed by the patch probe routing module 150 to route the data flow to the appropriate location(s), such as a storage location in archive 138, a display area at display system 134, and/or an insertion point within a report, among other things.

At step 212, the signal processor 132 of the ultrasound imaging system 100 may receive an indication to unassign the ultrasound probe 104a, 104b. For example, a patch probe unassignment module 160 of the signal processor 132 may detect the absence of the ultrasound probe 104a, 104b. The absence of the ultrasound probe 104a, 104b detected by the patch probe unassignment module 160 may be due to, for example, an ultrasound imaging system operator disconnecting the probe 104a, 104b from the ultrasound imaging system 100. The disconnection may be in response to an instruction provided to the ultrasound imaging system 100 via the user input module 130, powering off the ultrasound probe 104a, 104b, moving the ultrasound probe 104a, 104b out of a wireless range of the ultrasound imaging system 100, and/or breaking an electrical connection between the ultrasound probe 104a, 104b and the ultrasound imaging system 100, among other things. As another example, the patch probe unassignment module 160 of the signal processor may detect that the ultrasound probe 104a, 104b is acquiring non-relevant ultrasound image data. The non-relevant ultrasound image data detected by the patch probe unassignment module 160 may include, for example, an air-scanning condition and/or a detected patch probe movement condition. The air-scanning condition, representative of an ultrasound patch probe becoming unsecured from the patient, may be detected by the patch probe unassignment module 160 applying image analysis algorithms that determine, for example, that the acquired ultrasound image data is static over a predetermined period of time and/or that the only received reflections are from superficial gel. The patch probe movement condition, representative of an ultrasound patch probe 104a, 104b moving relative to the surface of the skin of the patient such that the probe is no longer acquiring ultrasound image data of the relevant patient anatomy, may be detected by the patch probe unassignment module 160 analyzing, for example, motion sensor data from motion sensors connected to the ultrasound patch probe 104a, 104b and the patient. As another example, the patch probe movement condition may be detected by the patch probe unassignment module 150 analyzing the superficial area of the acquired ultrasound image data and comparing sequential frames using cross-correlation algorithms to detect ultrasound probe motion.

At step 214, the ultrasound imaging system 100 may stop acquiring and/or routing ultrasound image data from the ultrasound probe 104a, 104b unassigned at step 212. For example, the patch probe unassignment module 160 may instruct an ultrasound probe 104a, 104b to stop acquiring ultrasound image data and/or may instruct the patch probe routing module 150 to stop routing a data flow associated with an unassigned ultrasound probe 104a, 104b. In various embodiments, the instructions from the patch probe unassignment module 160 to stop acquiring and stop routing may be substantially simultaneous. In certain embodiments, the patch probe unassignment module 160 may continue acquiring but may stop routing ultrasound image data flows from ultrasound patch probes 104a, 104b if an air scanning or probe movement condition is identified at step 212. The patch probe unassignment module 160 may re-assign the patch probe 104a, 104b if, for example, the patch probe 104a, 104b is reattached and/or repositioned within a predetermined period of time so that the patch probe routing module 150 may resume routing the data flow. Otherwise, the patch probe unassignment module 160 may turn off and/or disconnect the patch probe 104a, 104b after the predetermined period of time of continuous air scanning or non-resolved probe movement. The method may return to step 202 to reconnect and/or reassign an unassigned ultrasound probe 104a, 104b after the probe 104a, 104b is unassigned by the patch probe unassignment module 160 or after the probe 104a, 104b is unassigned by the patch probe unassignment module 160 and a predetermined period of time has elapsed.

Aspects of the present disclosure provide a method 200 and system 100 for assigning, routing, and unassigning data flows of ultrasound patch probes 104a, 104b. In accordance with various embodiments, the method 200 comprises detecting 204, by a signal processor 132, 140 of an ultrasound imaging system 100, a presence of an ultrasound patch probe 104a, 104b to establish a connection between the ultrasound patch probe 104a, 104b and the ultrasound imaging system 100. The method 200 comprises assigning 206, by the signal processor 132, 140, the detected ultrasound patch probe 104a, 104b to one or both of a patient and an anatomy of the patient. The method 200 comprises mapping 208, by the signal processor 132, 140, the ultrasound patch probe 104a, 104b to ultrasound image routing parameters based on the assignment. The method 200 comprises routing 210, by the signal processor 132, 150, ultrasound image data acquired by the ultrasound patch probe 104a, 104b to one or both of a display area of a display system 134 for presentation of the acquired ultrasound image data and a storage location of a data storage medium 138 for storing the acquired ultrasound image data based on the ultrasound image routing parameters.

In various embodiments, the method 200 comprises mapping 208, by the signal processor 132, 140, the ultrasound patch probe 104a, 104b to a pre-defined set of ultrasound scanning parameters based on the assignment. In certain embodiments, the method 200 comprises continuously acquiring 210 the ultrasound image data by the ultrasound patch probe 104a, 104b based at least in part on the pre-defined set of ultrasound scanning parameters. In an exemplary embodiment, the connection between the ultrasound patch probe 104a, 104b and the ultrasound imaging system 100 is a wireless connection. In a representative embodiment, the assignment of the detected ultrasound patch probe 104a, 104b is based at least in part on user assignment settings input via an assignment dialog user interface.

In certain embodiments, the method 200 comprises receiving 212, by the signal processor 132, 160, an indication to unassign the ultrasound patch probe 104a, 104b. In an exemplary embodiment, the indication is an air-scanning condition detected by the signal processor 132, 160 applying image analysis algorithms that determine if one or both of the acquired ultrasound image data is static over a predetermined period of time, and the acquired ultrasound image data consists of reflections from superficial gel. In various embodiments, the indication is a patch probe movement condition detected by the signal processor 132, 160 one or both of applying cross-correlation algorithms that compare a superficial area of the acquired ultrasound image data in sequential frames to determine if the ultrasound patch probe 104a, 104b has moved relative to a skin surface of the patient, and analyzing motion sensor data received from motion sensors attached to the ultrasound patch probe 104a, 104b and the patient to determine if the ultrasound patch probe 104a, 104b has moved relative to a skin surface of the patient. In a representative embodiment, the method 200 comprises, in response to the received indication to unassign the ultrasound patch probe 104a, 104b, one or both of stopping 214 acquiring ultrasound image data from the unassigned ultrasound patch probe 104a, 104b, and stopping 214 routing ultrasound image data received at the signal processor 132 from the unassigned ultrasound patch probe 104a, 104b.

Various embodiments provide a system 100 comprising an ultrasound patch probe 104a, 104b and an ultrasound imaging system 100 having a signal processor 132, 140, 150, 160. The ultrasound patch probe 104a, 104b includes ultrasound image acquisition functionality. The ultrasound patch probe 104a, 104b is configured to be selectively placed on a patient in communication range of the ultrasound imaging system 100. The signal processor 132, 140 is configured to detect a presence of the ultrasound patch probe 104a, 104b to establish a connection between the ultrasound patch probe 104a, 104b and the ultrasound imaging system 100. The signal processor 132, 140 is configured to assign the detected ultrasound patch probe 104a, 104b to one or both of a patient and an anatomy of the patient. The signal processor 132, 140 is configured to map the ultrasound patch probe 104a, 104b to ultrasound image routing parameters based on the assignment. The signal processor 132, 150 is configured to route ultrasound image data acquired by the ultrasound patch probe 104a, 104b based on the ultrasound image routing parameters to one or both of a display area of a display system 134 for presentation of the acquired ultrasound image data, and a storage location of a data storage medium 138 for storing the acquired ultrasound image data.

In an exemplary embodiment, the signal processor 132, 140 is configured to map the ultrasound patch probe 104a, 104b to a pre-defined set of ultrasound scanning parameters based on the assignment. The ultrasound image data is acquired by the ultrasound patch probe 104a, 104b based at least in part on the pre-defined set of ultrasound scanning parameters. In various embodiments, the signal processor 132, 160 is configured to receive an indication to unassign the ultrasound patch probe 104a, 104b. In certain embodiments, the indication is an air-scanning condition detected by the signal processor 132, 160 applying image analysis algorithms that determine if one or both of the acquired ultrasound image data is static over a predetermined period of time, and the acquired ultrasound image data consists of reflections from superficial gel. In a representative embodiment, the indication is a patch probe movement condition detected by the signal processor 132, 160 one or both of applying cross-correlation algorithms that compare a superficial area of the acquired ultrasound image data in sequential frames to determine if the ultrasound patch probe 104a, 104b has moved relative to a skin surface of the patient, and analyzing motion sensor data received from motion sensors attached to the ultrasound patch probe 104a, 104b and the patient to determine if the ultrasound patch probe 104a, 104b has moved relative to a skin surface of the patient. In an exemplary embodiment, the signal processor 132, 150, 160 is operable to one or both of instruct the unassigned ultrasound patch probe 104a, 104b to stop acquiring ultrasound image data, and stop routing ultrasound image data received from the unassigned ultrasound patch probe 104a, 104b.

Certain embodiments provide a non-transitory computer readable medium having a stored thereon, a computer program having at least one code section that is executable by a machine for causing the machine to perform steps 200 disclosed herein. Exemplary steps 200 may comprise detecting 204 a presence of an ultrasound patch probe 104a, 104b to establish a connection between the ultrasound patch probe 104a, 104b and an ultrasound imaging system 100. The steps 200 may comprise assigning 206 the detected ultrasound patch probe 104a, 104b to one or both of a patient and an anatomy of the patient. The steps 200 may comprise mapping 208 the ultrasound patch probe 104a, 104b to ultrasound image routing parameters based on the assignment. The steps 200 may comprise routing 210 ultrasound image data acquired by the ultrasound patch probe 104a, 104b based on the ultrasound image routing parameters to one or both of a display area of a display system 134 for presentation of the acquired ultrasound image data, and a storage location of a data storage medium 138 for storing the acquired ultrasound image data.

In a representative embodiment, the steps 200 may comprise mapping 208 the ultrasound patch probe 104a, 104b to a pre-defined set of ultrasound scanning parameters based on the assignment. In various embodiments, the steps 200 may comprise continuously acquiring 210 the ultrasound image data based at least in part on the pre-defined set of ultrasound scanning parameters. In an exemplary embodiment, the steps 200 may comprise receiving 212 an indication to unassign the ultrasound patch probe 104a, 104b. In certain embodiments, in response to the received indication to unassign the ultrasound patch probe 104a, 104b, the steps 200 may comprise one or both of stopping acquiring 214 ultrasound image data from the unassigned ultrasound patch probe 104a, 104b, and stopping routing 214 ultrasound image data received from the unassigned ultrasound patch probe 104a, 104b.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for assigning, routing, and unassigning data flows of ultrasound patch probes 104a, 104b.

Accordingly, various embodiments may be realized in hardware, software, or a combination of hardware and software. Various embodiments may be realized in a centralized fashion in at least one computer system, or in a distributed fashion where different elements are spread across several interconnected computer systems. Any kind of computer system or other apparatus adapted for carrying out the methods described herein is suited. A typical combination of hardware and software may be a general-purpose computer system with a computer program that, when being loaded and executed, controls the computer system such that it carries out the methods described herein.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While various embodiments have been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method, comprising:
detecting, by a signal processor of an ultrasound imaging system, a presence of an ultrasound patch probe to establish a connection between the ultrasound patch probe and the ultrasound imaging system;
assigning, by the signal processor, the detected ultrasound patch probe to one or both of a patient and an anatomy of the patient;
mapping, by the signal processor, the ultrasound patch probe to ultrasound image routing parameters based on the assignment; and
routing, by the signal processor, ultrasound image data acquired by the ultrasound patch probe to one or both of a display area of a display system for presentation of the acquired ultrasound image data and a storage location of a data storage medium for storing the acquired ultrasound image data based on the ultrasound image routing parameters.

2. The method of claim 1, comprising mapping, by the signal processor, the ultrasound patch probe to a pre-defined set of ultrasound scanning parameters based on the assignment.

3. The method of claim 2, comprising continuously acquiring the ultrasound image data by the ultrasound patch probe based at least in part on the pre-defined set of ultrasound scanning parameters.

4. The method of claim 1, wherein the connection between the ultrasound patch probe and the ultrasound imaging system is a wireless connection.

5. The method of claim 1, wherein the assignment of the detected ultrasound patch probe is based at least in part on user assignment settings input via an assignment dialog user interface.

6. The method of claim 1, comprising receiving, by the signal processor, an indication to unassign the ultrasound patch probe.

7. The method of claim 6, wherein the indication is an air-scanning condition detected by the signal processor applying image analysis algorithms that determine if one or both of:
the acquired ultrasound image data is static over a predetermined period of time, and
the acquired ultrasound image data consists of reflections from superficial gel.

8. The method of claim 6, wherein the indication is a patch probe movement condition detected by the signal processor one or both of:
applying cross-correlation algorithms that compare a superficial area of the acquired ultrasound image data in sequential frames to determine if the ultrasound patch probe has moved relative to a skin surface of the patient, and
analyzing motion sensor data received from motion sensors attached to the ultrasound patch probe and the patient to determine if the ultrasound patch probe has moved relative to a skin surface of the patient.

9. The method of claim 6, comprising, in response to the received indication to unassign the ultrasound patch probe, one or both of:
stopping acquiring ultrasound image data from the unassigned ultrasound patch probe, and
stopping routing ultrasound image data received at the signal processor from the unassigned ultrasound patch probe.

10. A system, comprising:
an ultrasound patch probe having ultrasound image acquisition functionality, wherein the ultrasound patch probe is configured to be selectively placed on a patient in communication range of an ultrasound imaging system; and
the ultrasound imaging system comprising:
a signal processor configured to:
detect a presence of the ultrasound patch probe to establish a connection between the ultrasound patch probe and the ultrasound imaging system;
assign the detected ultrasound patch probe to one or both of a patient and an anatomy of the patient;
map the ultrasound patch probe to ultrasound image routing parameters based on the assignment; and
route ultrasound image data acquired by the ultrasound patch probe based on the ultrasound image routing parameters to one or both of:
a display area of a display system for presentation of the acquired ultrasound image data, and
a storage location of a data storage medium for storing the acquired ultrasound image data.

11. The system of claim 10, wherein the signal processor is configured to map the ultrasound patch probe to a pre-defined set of ultrasound scanning parameters based on the assignment, and wherein the ultrasound image data is acquired by the ultrasound patch probe based at least in part on the pre-defined set of ultrasound scanning parameters.

12. The system of claim 10, wherein the signal processor is configured to receive an indication to unassign the ultrasound patch probe.

13. The system of claim 12, wherein the indication is an air-scanning condition detected by the signal processor applying image analysis algorithms that determine if one or both of:
the acquired ultrasound image data is static over a predetermined period of time, and
the acquired ultrasound image data consists of reflections from superficial gel.

14. The system of claim 12, wherein the indication is a patch probe movement condition detected by the signal processor one or both of:
applying cross-correlation algorithms that compare a superficial area of the acquired ultrasound image data in sequential frames to determine if the ultrasound patch probe has moved relative to a skin surface of the patient, and
analyzing motion sensor data received from motion sensors attached to the ultrasound patch probe and the patient to determine if the ultrasound patch probe has moved relative to a skin surface of the patient.

15. The system of claim 12, wherein the signal processor is operable to one or both of:
instruct the unassigned ultrasound patch probe to stop acquiring ultrasound image data, and
stop routing ultrasound image data received from the unassigned ultrasound patch probe.

16. A non-transitory computer readable medium having stored thereon, a computer program having at least one code section, the at least one code section being executable by a machine for causing the machine to perform steps comprising:
detecting a presence of an ultrasound patch probe to establish a connection between the ultrasound patch probe and an ultrasound imaging system;
assigning the detected ultrasound patch probe to one or both of a patient and an anatomy of the patient;
mapping the ultrasound patch probe to ultrasound image routing parameters based on the assignment; and
routing ultrasound image data acquired by the ultrasound patch probe based on the ultrasound image routing parameters to one or both of:
a display area of a display system for presentation of the acquired ultrasound image data, and
a storage location of a data storage medium for storing the acquired ultrasound image data.

17. The non-transitory computer readable medium according to claim 16, comprising mapping the ultrasound patch probe to a pre-defined set of ultrasound scanning parameters based on the assignment.

18. The non-transitory computer readable medium according to claim 17, comprising continuously acquiring the ultrasound image data based at least in part on the pre-defined set of ultrasound scanning parameters.

19. The non-transitory computer readable medium according to claim 16, comprising receiving an indication to unassign the ultrasound patch probe.

20. The non-transitory computer readable medium according to claim 19, comprising, in response to the received indication to unassign the ultrasound patch probe, one or both of:
stopping acquiring ultrasound image data from the unassigned ultrasound patch probe, and
stopping routing ultrasound image data received from the unassigned ultrasound patch probe.
